(19) United States
(12) Patent Application Publication
(10) Pub. No.: US 2026/0251566 A1
(43) Pub. Date: Aug. 27, 2026

(54) ACTIVATABLE ENVIRONMENTAL INDICATORS

(71) Applicant: Zebra Technologies Corporation, Lincolnshire, IL (US)

(72) Inventors: Eric W. LIBERATO, Pequannock, NJ (US); Mohannad ABDO, Clifton, NJ (US); Anthony J. CECCHIN, Kildeer, IL (US)

(73) Assignee: Zebra Technologies Corporation, Lincolnshire, IL (US)

(21) Appl. No.: 19/065,218

(22) Filed: Feb. 27, 2025

(51) Int. Cl. G01N 21/47; G01N 15/08; G01N 33/00

(52) U.S. Cl. CPC G01N 21/47; G01N 15/088; G01N 33/0027; G01N 2015/0846

(57) ABSTRACT

Methods of making an environmental indicator are described. The indicators described herein comprise a layer of cavitated porous light scattering material. The cavitated porous light scattering material includes particles that scatter light due to spaces between the particles, and further, the particles are themselves made of a porous material that scatters light.

### BACKGROUND

Many commercial products are sensitive to overly high or low temperatures or humidity or the presence of certain gases (e.g., oxygen, ammonia, ethylene, etc.). Exposure to such high or low temperatures, humidity or gas concentrations, and/or extended periods of time at elevated temperatures, humidity or gas levels may cause a product to spoil or lose efficacy/quality. Often commercially available indicators are pre-fabricated to work for ranges of temperatures or ranges of humidity or gas concentrations and a user does not have the ability to customize the indicators.

There is a need for indicators that can be easily customized for detecting a desired range of temperature, humidity or gas, depending on a user's needs.

### SUMMARY

In some embodiments, provided is a method of making an environmental indicator, some steps in the method being:
providing a substrate;
providing an indicia in an indicator region on the substrate;
providing a reservoir on or in the substrate;
overlaying cavitated porous light scattering material on the substrate, the light scattering material masking the indica;
closing the pores of the cavitated porous light scattering material in a boundary region by heating portions of the cavitated porous light scattering material, the boundary region defining a channel through the porous light scattering material between the reservoir and the indicator region;
and
adding indicator material to the reservoir,
   the indicator material configured to liquify responsive to a predetermined environmental stimulus and flow through the channel to the indicator region filling the cavities in the cavitated porous light scattering material in the indicator region and thereby unmasking the indicia.

In some embodiments of the method, the material in the reservoir may be a polyalkyl acrylate, an alkane wax, and/or combinations thereof.

In some embodiments of the method, the polyalkyl acrylate is a side chain crystalline (SCC) polymer having C₁₀-C₃₀ alkyl side chains.

In some embodiments of the method, the alkane wax may be a C₁₈-C₄₀ hydrocarbon, or a mixture of C₁₈-C₄₀ hydrocarbons.

In some embodiments of the method, the material in the reservoir may have a plurality of microcapsules which have a liquid or a liquefiable material microencapsulated in a frangible shell, wherein the frangible shells are configured to release the liquid or the liquefiable material responsive to an application of an activation action.

In some embodiments of the method, an activating action is application of a compressive force or application of heat to the reservoir.

In some embodiments of the method, some additional steps in the method may be:
a plurality of needles configured to penetrate the channel, placing the reservoir into fluid communication with the channel.

In some embodiments of the method, the needles are hollow and the material in the reservoir flows through the needles.

In some embodiments of the method, an indicium is a machine-readable indicium, which is readable through the cavitated porous light scattering material after the liquefiable material fills the pores in the channel and is unreadable through the cavitated porous light scattering material before the liquefiable material fills the pores in the channel.

In some embodiments of the method, the environmental stimulus is selected from an exposure to an ambient temperature above a predetermined high temperature threshold, an exposure to an ambient temperature above a predetermined high temperature threshold for at least a predetermined amount of time, a cumulative exposure to ambient heat exceeding a predetermined cumulative heat threshold, a cumulative exposure to humidity exceeding a predetermined cumulative humidity exposure threshold, and/or a cumulative exposure to a gas exceeding a predetermined cumulative gas exposure threshold.

In some embodiments of the method, the cavitated porous light scattering material may be a material selected from a side chain (SCC) crystalline polymer, Polytetrafluoroethylene (PTFE), Ultra High Molecular Weight Polyethylene (UHMW PE), Ultra High Molecular Weight Polypropylene (UHMW PP), High Density Polyethylene (HDPE), Polyvinylidene Fluoride (PVDF), Perfluoroalkoxy alkane (PFA), Liquid Crystalline Polymer (LCP), or combinations thereof.

In some embodiments of the method, the cavitated porous light scattering material may be a side chain crystalline polymer.

In some embodiments of the method, the cavitated porous light scattering material has particles having an average diameter ranging from about 200 nm to about 800 nm.

In some embodiments of the method, the cavitated porous light scattering material has particles having an average diameter ranging from about 400 nm to about 600 nm.

In some embodiments of the method, portions of the cavitated porous light scattering material are heated (e.g., above the melting temperature of the material, e.g., using a direct thermal printer heating element, laser heating, direct contact with a heating element, etc.), thereby closing the pores of the cavitated porous light scattering material in a boundary region.

In some embodiments of the method, portions of the cavitated porous light scattering material are heated (e.g., above the melting temperature of the material, e.g., using a direct thermal printer heating element, laser heating, direct contact with a heating element, etc.) to temperatures ranging from about 40° C. to about 300° C.

In some embodiments, provided herein is an environmental indicator prepared by any method described above and herein.

In some embodiments, provided is an environmental indicator having:
a substrate;
an indicia in an indicator region on the substrate;
a layer of overlay material overlaying the substrate,
   a first portion of the layer of overlay material over the indicator region being cavitated, porous and light scattering, and masking the indicia;
   a second portion of the layer of overlay material being cavitated, porous and light scattering, and forming a channel connecting the indicator region with the reservoir;
   a third portion of the layer of overlay material having closed pores to define the boundaries of the channel;
and
a reservoir of indicator material;
   the indicator material configured to liquify responsive to a predetermined environmental stimulus and flow through the channel to the indicator region filling the cavities in the cavitated porous light scattering material in the indicator region and thereby unmasking the indicia.

In some embodiments of the environmental indicator, the material in the reservoir may be a polyalkyl acrylate, an alkane wax, and/or combinations thereof.

In some embodiments of the environmental indicator, the polyalkyl acrylate is a side chain crystalline (SCC) polymer having C₁₀-C₃₀ alkyl side chains.

In some embodiments of the environmental indicator, the alkane wax may be a C₁₈ C₄₀ hydrocarbon, or a mixture of C₄₀-C₄₀ hydrocarbons.

In some embodiments of the environmental indicator, the material in the reservoir may be a plurality of microcapsules which have a liquid or a liquefiable material microencapsulated in a frangible shell, wherein the frangible shells are configured to release the liquid or the liquefiable material responsive to an application of an activation action.

In some embodiments of the environmental indicator, an activating action is application of a compressive force or application of heat to the reservoir.

In some embodiments, the environmental indicator may further have: a plurality of microneedles configured to penetrate the channel, placing the reservoir into fluid communication with the channel.

In some embodiments, the needles are hollow and the material in the reservoir flows through the needles.

In some embodiments of the environmental indicator, an indicium is a machine-readable indicium, which is readable through the cavitated porous light scattering material after the liquefiable material fills the pores in the channel and is unreadable through the cavitated porous light scattering material before the liquefiable material fills the pores in the channel.

In some embodiments of the environmental indicator, the environmental stimulus may be an ambient temperature above a predetermined high temperature threshold, an exposure to an ambient temperature above a predetermined high temperature threshold for at least a predetermined amount of time, a cumulative exposure to ambient heat exceeding a predetermined cumulative heat threshold, a cumulative exposure to humidity exceeding a predetermined cumulative humidity exposure threshold, and a cumulative exposure to a gas exceeding a predetermined cumulative gas exposure threshold.

In some embodiments of the environmental indicator, the cavitated porous light scattering material may be a material selected from a side chain crystalline polymer, Polytetrafluoroethylene (PTFE), Ultra High Molecular Weight Polyethylene (UHMW PE), Ultra High Molecular Weight Polypropylene (UHMW PP), High Density Polyethylene (HDPE) Polyvinylidene Fluoride (PVDF), Perfluoroalkoxy alkane (PFA), Liquid Crystalline Polymer (LCP), or combinations thereof.

In some embodiments of the environmental indicator, the cavitated porous light scattering material may be a side chain crystalline polymer.

In some embodiments of the environmental indicator, the cavitated porous light scattering material has particles having an average diameter ranging from about 200 nm to about 800 nm.

In some embodiments of the environmental indicator, the cavitated porous light scattering material has particles having an average diameter ranging from about 400 nm to about 600 nm.

In some embodiments of the environmental indicator, portions of the cavitated porous light scattering material are heated (e.g., above the melting temperature of the material, e.g., using a direct thermal printer heating element, laser heating, direct contact with a heating element, etc.), thereby closing the pores of the cavitated porous light scattering material to define the boundaries of the channel.

In some embodiments of the environmental indicator, portions of the cavitated porous light scattering material are heated (e.g., above the melting temperature of the material, e.g., using a direct thermal printer heating element, laser heating, direct contact with a heating element, etc.) to temperatures ranging from about 40° C. to about 300° C.

### BRIEF DESCRIPTION OF THE DRAWINGS

The accompanying figures, where like reference numerals refer to identical or operationally similar elements throughout the separate views, together with the detailed description below, are incorporated in and form part of the specification, and serve to further illustrate embodiments of concepts that include the claimed invention, and explain various principles and advantages of those embodiments.

FIG. 1A depicts a top view of an example embodiment of an indicator.

FIG. 1B depicts a cross section view of the indicator of FIG. 1A.

FIG. 2 depicts activation of an environmental indicator of FIG. 1A and revealing of the indicium.

FIG. 3 depicts a further example embodiment of an environmental indicator.

FIG. 4 depicts an example embodiment for an environmental indicator, and activation and revealing of the indicium.

FIG. 5 depicts a cross section and a first example embodiment for the indicator of FIG. 4.

FIG. 6 depicts a cross section and a second example embodiment for the indicator of FIG. 4.

FIG. 7 depicts an example embodiment of a covered indicator.

FIG. 8 is a photograph depicting results from the experiment described in Example 1.

FIG. 9 depicts a flow chart showing preparation of indicators described herein, customizing the indicators, and using the indicators according to embodiments of the disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Commercially available indicator labels often have an inflexible design where the indicator label is pre-fabricated and can only be used for manufacturer-provided ranges of threshold temperatures, or ranges of threshold levels of humidity or levels of gas, or time of exposure required for a particular exposure to trigger an indicator, e.g., time-temperature exposure indication. By contrast, indicators described herein can be tailored to a user's needs, e.g., for a user-selected temperature range, or a user-selected timer or time of exposure feature.

The indicators described herein have a layer of cavitated porous light scattering material. The cavitated porous light scattering material may be crystalline or semi-crystalline, or may have particles that scatter light due to voids between the particles. Further, the particles may themselves be made of a porous material that scatters light. The light scattering materials appear opaque, and may obscure a dark background or indicia behind or beneath the light scattering materials. The light scattering material can be rendered transparent or translucent in several different processes. For example, the light scattering material can be melted, so that it resolidifies without the voids which cause light scattering. Alternatively, or in addition, a liquid may be used to fill gaps between particles or pores in the material.

If a portion of the light scattering material is subjected to a temperature above its melting point (e.g., by use of a thermal printer), the material in that portion can melt. By selectively melting the light scattering material, embodiments of the present disclosure can form a transparent or translucent boundary around an area of the light scattering material which can still scatter light. This allows for forming customizable boundaries in an indicator label depending on the user's requirements. The light scattering area circumscribed by the boundary region may form an indicator region where the light scattering material initially masks the indicium. Boundaries may also be formed to create channels through the light scattering material, e.g., providing fluid communication through the material between a reservoir of liquifiable material and the indicator region. This channel may be short and direct, or long and winding, allowing the length of the channel and the time needed for material to flow from the reservoir to the indicator region to be customized.

The distance traversed by the ingressed fluid can be customized by varying the length or the thickness of the channel through the light scattering material. Thus, in addition to masking or unmasking of the indicium, the indicator can also incorporate a timer feature where the distance and/or depth traversed by the ingressed fluid can correspond to the duration of exposure to a temperature above a high temperature excursion threshold, or exposure to humidity, or gas.

The terms “a” and “an” are defined as one or more unless explicitly stated otherwise herein.

The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. “Cavitated porous light scattering material” or “light scattering material” or “light scattering layer” refers to material that has cavities or spaces or voids which scatter light. In some embodiments, cavitated porous light scattering material may be crystalline or semicrystalline. In some embodiments, cavitated porous light scattering material may have particles with cavities or spaces or voids between particles, and the particles themselves are porous which also allows for light scattering. “Indicator region” or “indicator area” refers to a portion of an indicator where an observable change indicating exposure to an environmental stimulus above a predetermined threshold is produced. In some embodiments, the indicator region may be overlayed on and may overlap fully, or in part, with the indicium. “Channel” refers to an area that facilitates fluid communication between a reservoir and an indicator area.

The term “polymer”, and its linguistic variations, refers to copolymers, and higher order polymers, as well as homopolymers, unless the context indicates otherwise, for example, by describing or referencing one or more specific homopolymers.

“Polyalkyl acrylates” or “polyalkyl acrylate polymers” refers to homopolymers or copolymers of n-alkyl acrylates. In some embodiments, polyalkyl acrylates include side chain crystalline (SCC) polymers and liquid crystal polymers (LCPs) (including side chain liquid crystal polymers (SCLCPs)). In some embodiments, SCC polymers or LCPs may have the same side chain (homopolymers). In some embodiments, SCC polymers or LCPs may have different side chains (copolymers of different monomers).

“Alkane wax,” in some embodiments, may be a paraffin wax which may be a mixture of hydrocarbons. In some embodiments, alkane wax may be an alkane which is a solid and melts responsive to exposure to a temperature above a predetermined high temperature excursion threshold.

“Hydrocarbons” are compounds consisting of carbon and hydrogen. Examples include and are not limited to alkanes, alkenes, alkynes, cycloalkanes, and cycloalkenes. Hydrocarbons may be saturated or unsaturated hydrocarbons. Hydrocarbons may be straight chain or branched hydrocarbons. Hydrocarbons may be acyclic or cyclic (including polycyclic and aromatic).

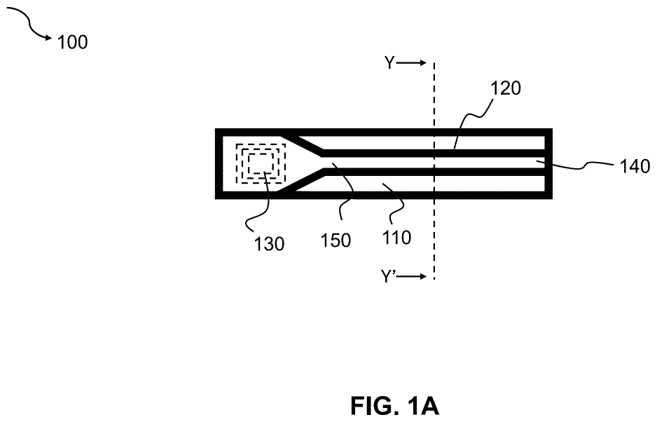

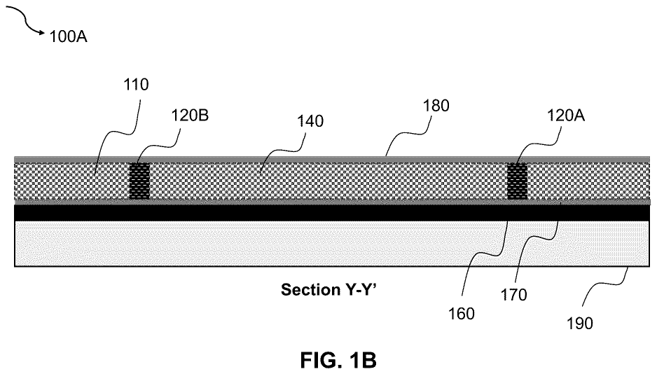

FIG. 1A shows a top view of an embodiment of an environmental indicator 100. FIG. 1B shows a cross section of the environmental indicator 100. As shown in FIG. 1A, a light scattering material 110 is overlayed on a substrate. A border region 120 is created by subjecting portions of the light scattering material to a high temperature, causing the material to melt, and resolidify in a form that is no longer light scattering. Initially, the environmental indicator 100 can be devoid of the border region 120 and the border region 120 can be created in a pattern chosen by a user, for example by using a thermal printer. In the present example, the border region 120 forms a perimeter for the indicator region 140. As the border region 120 can be specified or customized, the shape and size of the border region 120 can vary. A reservoir 130 is in fluid communication with the indicator region 140, e.g., through the cavitated layer via a channel 150. As an example, the border region 120 can encompass the reservoir 120 or the border region 120 can be open to the reservoir so that the reservoir 130 is in fluid communication with the indicator region 140 defined by the border region 120. The entire structure may be covered by a sealing layer, which, e.g., may be opaque except having a transparent window. The reservoir 130 may be covered by an optional sealing layer and may optionally have perforations in the sealing layer. The dashed line Y-Y′ represents the plane of the cross section which is shown in FIG. 1B.

As shown in FIG. 1B, a substrate 190 is overlayed with a pigment 160. An optional sealing layer 170 is overlayed on the pigment layer 160. In an alternate embodiment, the substrate 190 might itself be a colored substrate or, e.g., a laser etched substrate having an indicium, and an optional sealing layer 170 is overlayed on the colored substrate. A cavitated porous light scattering material 110 is overlayed on the substrate 190, or on the pigment layer 160 if present, or on the sealing layer 170, if present. A further optional sealing layer 180 is overlayed on the light scattering material 110. The light scattering material appears opaque, and white or light colored, and may obscure, mask or hide a dark colored pigment or other indicia behind the light scattering material. The reservoir 130 is not shown in this cross section.

Portions of the light scattering material, e.g., 120A and 120B, are subjected to a high temperature so that the portions 120A and 120B of the light scattering material undergo melting, then resolidify in a form that is transparent or translucent, and the color of the pigment or the colored substrate is revealed to provide a border region. The heating is to a point above the melting point of the light scattering material, for a sufficient time for the material to melt. It may be applied in a variety of manners, e.g., using a direct thermal printer heating element (e.g., a thermal printhead), laser heating, direct contact with a heating element, etc. The border region forms an impermeable barrier around an indicator region 140 of the light scattering material. The border region is impermeable to fluid because the particles have melted and coalesced and do not allow fluid to flow through. The indicator region 140 defined by the border region masks the indicum because it is overlayed on the indicium (e.g., the pigment or the colored substrate). The indicator region 140 becomes transparent or translucent when fluid flows into the indicator region 140. The channel 150 is not shown in this cross section.

In some embodiments, the light scattering material includes spaces (e.g. voids) between particles which causes light scattering. Alternatively, or in addition, there may be space within the particles themselves, due to the morphology of the particles and/or their crystalline structure. The particles of the light scattering material can melt and coalesce (and/or eliminate voids within the particles) when subjected to a high temperature and the melted area becomes transparent or translucent after the material re-solidifies (and possibly while it is still liquid). Also, when a fluid flows into the spaces between particles or fills voids with the particles if they are porous, the light scattering material can become transparent or translucent.

The particles in the light scattering material may have regular shapes or irregular shapes. In some embodiments, the light scattering particles have a regular shape (e.g., beads or spheres) and an average diameter of about 200 nm to about 800 nm, about 200 nm to about 600 nm, or about 400 nm to about 600 nm. In other embodiments, the light scattering particles may have irregular shapes (e.g., irregular granules of varying shapes and sizes).

The light scattering material may be a liquifiable polymer chosen so that it can liquify in a desired temperature range, allowing for thermal creation of the boundary region. The temperature at which the boundary region is created may be the same, or may be different from the environmental temperature that the indicator is meant to detect. In other embodiments, the temperature at which the boundary region is created may be any temperature if the indicator is meant to detect humidity or a gas.

In some embodiments, the light scattering material may have particles of SCC polymers or LCPs (including side chain liquid crystal polymers (SCLCPs)). The melting temperature Tm of the SCC or LCP may be in the range of about 40° C. to about 300° C., and the SCC or LCP may be selected based on the temperature sensing indicator range of the indicator such that the SCC or LCP itself is not affected by the operating temperatures of the indicator.

In some embodiments, the light scattering material may be selected from thermoplastics such as Polytetrafluoroethylene (PTFE), Ultra High Molecular Weight Polyethylene (UHMW PE), Ultra High Molecular Weight Polypropylene (UHMW PP), High Density Polyethylene (HDPE), Polyvinylidene Fluoride (PVDF), or Perfluoroalkoxy alkane (PFA). In some embodiments, a thermoplastic used in the light scattering layer may be selected based on its melting temperature and the melting point of the thermoplastic is such that the thermoplastic itself is not affected by the operating temperatures of the indicator (e.g., the melting point of the thermoplastic is above (but not too far above) the temperature indication range.

The sealing layers 170 and 180 may be a variety of materials that protect the covered layers and prevent penetration of humidity or gas, e.g., polymeric films made of, for example, polyethylene terephthalate (PET), or biaxially oriented polypropylene (BOPP).

The substrate 190 may be manufactured from a variety of materials, e.g., paper, paperboard, cardboard, cotton, linen, jute, ramie, industrial hemp or rayon, polyamide, polyester, polyacrylate, polyurethanes or vinyl-based fibers, blended fibrous substrates based on cellulosic and non-cellulosic fibers; polymeric resin; and composites of polymeric resins with cellulosic or non-cellulosic fibrous material; and combinations thereof. The substrate may be a colored substrate, in which case a pigment may not be layered on the substrate. In some embodiments, the substrate may be laser etched (e.g., with a pattern for an indicium).

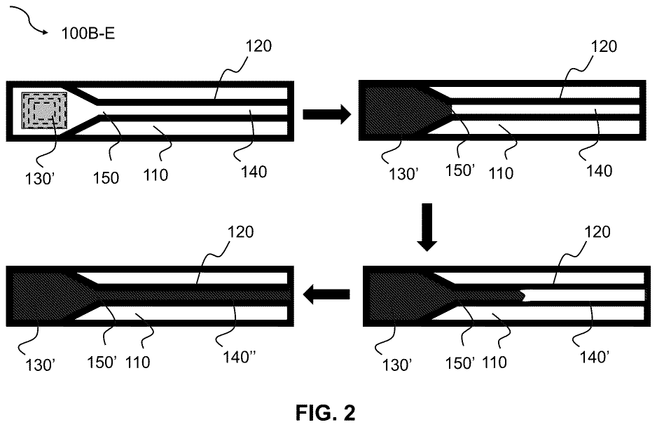

FIG. 2 shows a flow chart with a top view for activation of the indicator 100 of FIG. 1A. The material in the reservoir liquifies, 130′, in response to an environmental stimulus. The liquified material 130′ starts to flow from the reservoir via the channel 150′. The liquified material 130′ continues to flow from the reservoir, via the channel 150′, into the indicator area 140′. The liquified material in the reservoir 130′ flows through the channel 150′, to the indicator region 140″, the pigmented surface becomes unobscured in the indicator area 140″, thus revealing the indicium that had been masked by the light scattering material of the indicator region 140. In this embodiment, the reservoir is in or on the substrate and in proximity to the indicator region 140 on the substrate.

In some embodiments, the reservoir material is liquifiable when subjected to a threshold temperature. For instance, a reservoir material may have a polyalkyl acrylate, an alkane wax, or a combination thereof. When the indicator environment reaches a threshold temperature, the material in the reservoir melts and starts to flow into the indicator region as shown in FIG. 2.

Suitable liquefiable materials include waxes and synthetic polymeric materials that are solid below the threshold temperature and are, or can become, a flowing amorphous solid or a viscous liquid when at and/or above a threshold temperature. When solid, the synthetic polymeric material can be crystalline or partially crystalline. Crystalline or partially crystalline synthetic polymeric materials can have desirably sharp transitions from a solid state to a liquid state.

A liquifiable material may have a similar refractive index to the light scattering material. In this manner, when the liquefied material fills the voids of the light scattering layer, the light scattering layer (in the non-obscuring form) is rendered to be substantially transparent. In various examples, difference in the refractive index between the liquified material and the light scattering material of the light scattering layer is less than 0.5, less than 0.25, or less than 0.1.

In some embodiments, the reservoir material is liquifiable when subjected to a threshold humidity or a gas. For instance, a reservoir material may have activatable microcapsules and the reservoir is covered by an optional perforated sealing layer. When the indicator environment reaches a threshold humidity or temperature, the microcapsules burst or dissolve releasing the liquid in the microcapsules which starts to flow into the indicator region.

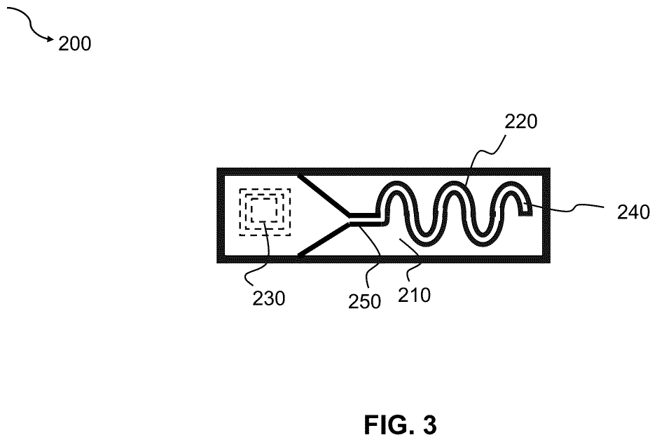

FIG. 3 shows a top view of an embodiment of an environmental indicator 200 where the indicator region′s shape and/or length can be changed to accommodate a timer feature, providing a time-temperature type activatable environmental exposure indicator. A light scattering material 210 is overlayed on the substrate. A border region 220 is created by subjecting portions of the light scattering material to a high temperature, causing the material to melt, and resolidify in a form that is no longer light scattering. The border region 220 can be created in a pattern chosen by a user, for example by using a direct thermal printer heating element, laser heating, direct contact with a heating element, etc., and as shown in this embodiment, the distance traversed by an ingressed fluid can be modified (e.g., for a timer feature) by changing the shape and/or length of the border region. The border region forms a perimeter for the indicator region 240. A reservoir 230 is in fluid communication with the indicator region 240, e.g., through the cavitated layer, via a channel 250. The entire structure may be covered by a sealing layer, which, e.g., may be opaque except having a transparent window. The reservoir may be covered by an optional sealing layer and may optionally have perforations in the sealing layer.

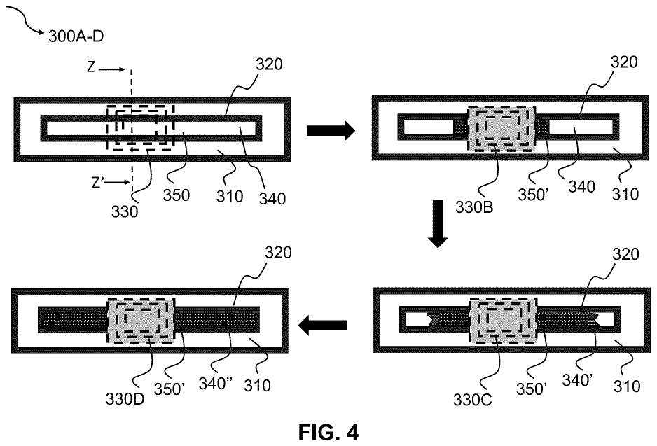

FIG. 4 shows a flow chart for a top view for an indicator 300 where the reservoir 330 is overlayed on the light scattering material 310 or may penetrate the light scattering material (e.g., via microneedles). The border region 320 defines an indicator area 340. When the material in the reservoir liquifies, 330′, in response to an environmental stimulus, the liquified material starts to flow from the reservoir to the indicator area 340 via the channel 350′. The liquified material from the reservoir, 330′, continues to flow through the indicator region 340′. The liquified material flows into the indicator region 340″ revealing the indicium that had been masked by the light scattering material of the indicator region.

For the indicators 100, 200, and 300, the length of the indicator region can be customized so that the distance traveled by the liquid through the indicator region can indicate the length of time that the indicator has exceeded a threshold temperature. In some embodiments, the width of the border region may be varied to accommodate a timer feature. For such a timer feature, the viscosity of the liquid moving through the indicator region can be chosen so that the distance traveled by the fluid through the indicator region can be calibrated to duration of time that the indicator has exceeded a threshold temperature or humidity or gas.

Viscosity for the liquid flowing through an indicator region depends on properties of the material in the reservoir such as molecular weight, or concentration (e.g., where a mixture of materials is used in the reservoir). In some embodiments, the material in the reservoir may be alkanes which may have a molten (liquified) viscosity of about 10 to about. 100 cP. In some embodiments, the material in the reservoir may be polalkyl acrylates which may have a molten (liquified) viscosity of about 500 cP to about 10000 cP. In some embodiments, the material in the reservoir may be hydrocarbons of lower molecular weight (e.g., less than 200 g/mol) which may have a molten (liquified) viscosity of about 0.3 cP to about 1 cP. In some embodiments, the material in the reservoir may be hydrocarbons of high molecular weight (e.g. more than 200 g/mol) which may have a molten (liquified) viscosity of about 20 cP to about 1000 cP. In some embodiments, the material in the reservoir may be side chain crystalline polymers which may have a molten (liquified) viscosity of about 500 cP to greater than about 10000 cP.

Depending on the viscosity of the liquified material, the rate at which the liquified material may move through the indicator region may range from about 1 cm to about 5 cm per hour for less viscous material, from about 0.1 cm to about 0.5 cm per hour, or even slower, for higher viscosity materials.

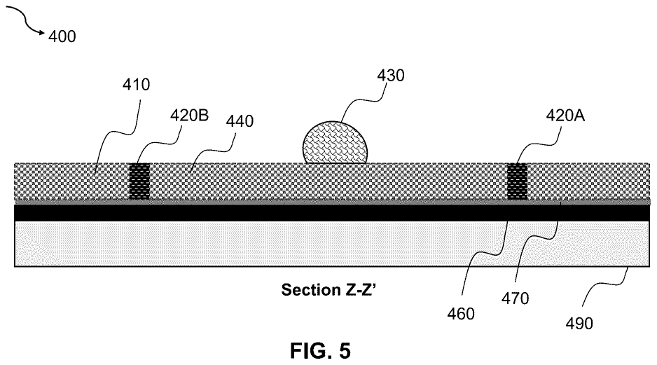

FIG. 5 shows a cross section of a first embodiment of FIG. 4 where the reservoir is overlayed on the light scattering layer. In some embodiments, the reservoir 430 may be a container (e.g., a plastic bubble) that can burst on application of a compressive force and release the liquid therein. In some embodiments, the reservoir 430 may be a container filled with a liquifiable material which liquifies when heat is applied to the reservoir. In this manner a user can control when the indicator 400 will start detecting an environmental condition.

A substrate 490 is overlayed with a pigment 460. An optional sealing layer 470 is overlayed on the pigment layer 460. In an alternate embodiment, the substrate 490 might itself be a colored substrate (e.g., the substrate is laser etched with an indicum) and an optional sealing layer 470 is overlayed on the colored substrate. A cavitated porous light scattering material 410 is overlayed on the substrate 490, or on the pigment layer 460 if present, or on the sealing layer 470, if present. The light scattering material appears opaque, and white or light colored, and may obscure, mask or hide a dark colored pigment or other indicia behind the light scattering material. A reservoir 430 filled with liquifiable material (e.g., a substance that can melt above a threshold temperature, or activatable microcapsules) is overlayed on the light scattering material 410. In this embodiment, there is no sealing layer between the reservoir 430 and the light scattering material 410.

Portions of the light scattering material, e.g., 420A and 420B, are subjected to a high temperature so that the portions 420A and 420B of the light scattering material undergo melting, then resolidify in a form that is transparent or translucent, and the color of the pigment or the colored substrate is revealed to provide a border region. The border region forms an impermeable barrier around an indicator area 440 of the light scattering material. The area 440 forms the indicator region and masks the indicum because it is overlayed on the indicium (e.g., the pigment or the colored substrate). The indicator area 440 becomes transparent or translucent when fluid flows into the indicator area from the reservoir 430.

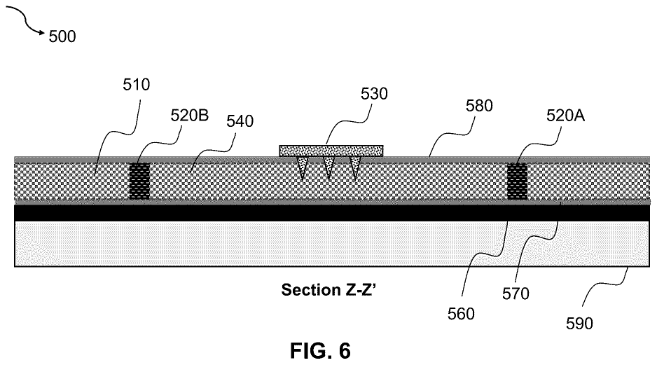

FIG. 6 shows a cross section of a second embodiment of FIG. 4 where the reservoir is overlayed on the light scattering layer. In FIG. 6, an indicator 500 has a reservoir 530 formed by a plurality of microneedles (only one microneedle is shown for clarity) overlayed on the light scattering layer. A substrate 590 is overlayed with a pigment 560. An optional sealing layer 570 is overlayed on the pigment layer 560. In an alternate embodiment, the substrate 590 might itself be a colored substrate (e.g., a laser etched substrate) and an optional sealing layer 570 is overlayed on the colored substrate. A cavitated porous light scattering material 510 is overlayed on the substrate 590, or on the pigment layer 560 if present, or on the sealing layer 570, if present. A further optional sealing layer 580 is overlayed on the light scattering material 510. The light scattering material appears opaque, and white or light colored, and may obscure, mask or hide a dark colored pigment or other indicia behind the light scattering material.

Portions of the light scattering material, e.g., 520A and 520B, are subjected to a high temperature so that the portions 520A and 520B of the light scattering material undergo melting, then resolidify in a form that is transparent or translucent, and the color of the pigment or the colored substrate is revealed to provide a border region as described above. The border region forms an impermeable barrier around an indicator area 540 of the light scattering material as described above. The area 540 forms the indicator region and masks the indicum because it is overlayed on the indicium (e.g., the pigment or the colored substrate). The indicator area 540 becomes transparent or translucent when fluid flows into the indicator area.

In some embodiments, for reservoir 530, the microneedles may be solid microneedles that can be wetted with a mixture of microcapsules. In some embodiments, the microneedles may be coated microneedles where the microneedles are pre-coated with a mixture of microcapsules. In some embodiments, the microneedles may be dissolving microneedles where the microneedles are filled or coated with a mixture of microcapsules and the microneedles will dissolve when exposed to an environmental stimulus (e.g., humidity) and release microcapsules. In some embodiments, the microneedles may be hollow microneedles that are filled with microcapsules, or any combination thereof. The solid microneedles can deliver a patch of microcapsules into the indicator region (e.g., by application of a compressive force and retracting the microneedle). The coated microneedles and the dissolving microneedles can place microcapsules into the indicator region and the microneedles need not be retracted. In response to an external stimulus (e.g., humidity, or temperature above a threshold temperature), the microcapsules release fluid into the indicator region. The hollow microneedles are filled with microcapsules which can be pushed into the indicator region (e.g., by application of a compression force) and the microneedles may, or may not be, retracted. Thus, needling delivers activatable microcapsules to the indicator region. The microcapsules are filled with liquid or a liquifiable material and may burst or melt in response to a compression force, or in response to a threshold temperature, humidity, or gas, thus delivering fluid to the indicator region, thereby exposing the indicium. According to some embodiments, some liquefiable materials may have a shared liquefaction point and solidification point, in which the liquefiable material liquefies and solidifies in about a temperature range less than 1 degree C. In other examples, the liquefiable materials discussed herein may exhibit hysteresis, where the solidification temperature is substantially lower (e.g., more than 1 degree C.) than the liquefication temperature. In some embodiments, the liquefaction and solidification temperatures are within about 0.1 degrees C., within about 0.5 degrees C., within about 1.0 degrees C., within about 1.5 degrees C., within about 2 degrees C., within about 2.5 degrees C., within about 3.0 degrees C., within about 3.5 degrees C., within about 4.0 degrees C., within about 4.5 degrees C., within about 5 degrees C., or within about 10 degrees C. of each other.

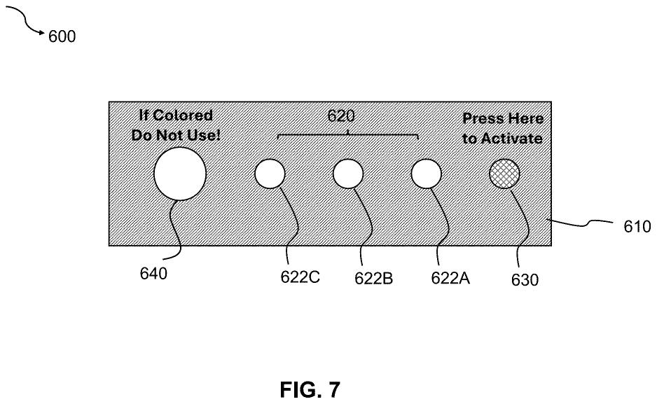

FIG. 7 shows a covered indicator (e.g., a commercial product). An activatable environmental exposure indicator 600 includes a cover layer 610 which overlays the indicator region and defines optional viewing windows 622A-C which indicate progression of fluid flow when a timer feature is included. The window 640 reveals a machine or human-readable indicium. The window 630 overlays the reservoir and the cover layer 610 optionally includes printed instructions for an activation action such as, e.g., “press here to activate” as illustrated in FIG. 7. The printed instructions may be printable through the cover layer, or may be printed on the cover layer.

In various embodiments, the indicia in the indicator 600 may be machine-readable indicia, such as a QR code or a barcode. In such embodiments, the machine-readable indicia may be unreadable, or unscannable when the light scattering layer is in the obscuring form, and the machine-readable indicia becomes readable or scannable when the light scattering layer is in the non-obscuring form.

In some examples the indicia are in the form of natural language text or symbols. In some examples, when the activatable environmental exposure indicator 600 is configured to be used with a host product having a spoiled temperature, the indicia may include, as a non-limiting example, the word “spoiled” which is initially obscured, and is then revealed when the activatable environmental exposure indicator is exposed to a temperature above a spoiling temperature for the host product.

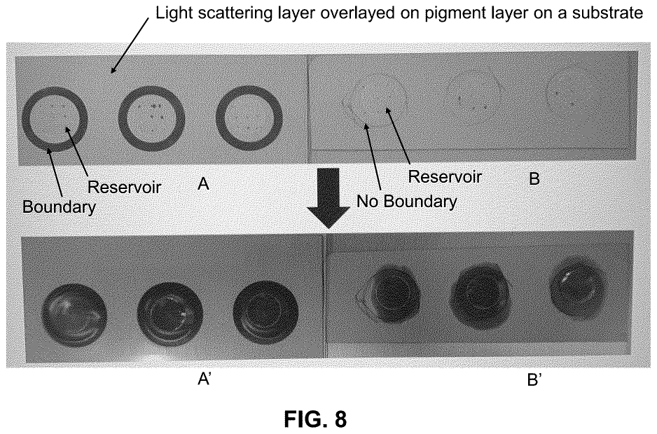

FIG. 8 shows results from an experiment described in Example 1. A border region was created in the light scattering material overlayed on a substrate. A reservoir of liquifiable material was placed in a reservoir and the liquifiable material was released into the area defined by the boundary region. FIG. 8 shows that the boundary region successfully prevented fluid from leaking out of the border region.

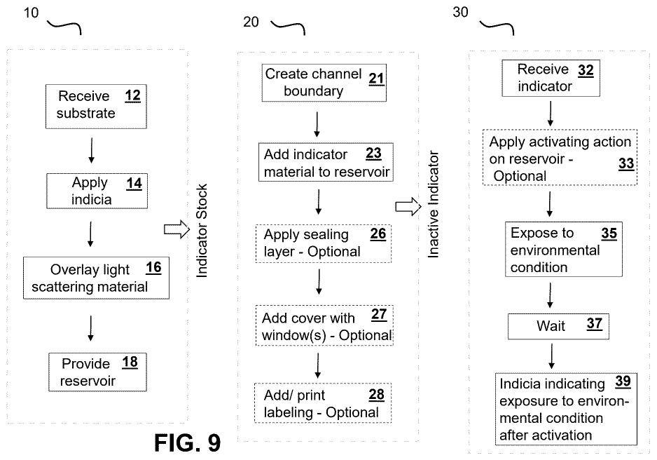

FIG. 9 illustrates a flowchart 10 of a first method for forming an indicator including blocks 12, 14, 16 and 18, a flowchart 20 of a second method for customizing an indicator including blocks 21, 23, 26, 27, and 28, and a flowchart 30 of a third method for deploying an indicator including blocks 32, 33, 35, 37, and 39. The method 10, the method 20, and the method 30 may be performed in the same or different locations, at the same or different times, and by the same or different actors.

Block 12 of the first method 10 describes receiving a substrate. For example, a substrate such as a substrate 190 described in FIG. 1B may be obtained or manufactured.

Block 14 of the first method describes applying indicia to the substrate. The indicia may be a pigmented area on the substrate, a printed word, numbering, symbol, or lettering, or a machine-readable symbol such as a bar code symbol, or any other predetermined indicia. The indicia may be printed, may be laser-etched, may be a pigment layer, or any other approach suitable for providing observable indicia. A manufacture stock of substrates with indica may be prepared.

Block 16 describes overlaying a cavitated porous light scattering material on the substrate. The light scattering material may overlay all or part of the substrate, and in particular may overlay the indicia. Because of its light scattering property, the light scattering material may mask the indicia so that it is either not visible at all, or not readable. The light scattering material may be dispensed on the substrate by various techniques such as powder dispensing, liquid dispensing, screen printing, flexo printing, gravure, slot die or ink jet printing methods. For example, screen printing may be achieved by preparing a composition of polymer particles (e.g., microspheres), additives including a surfactant, dispersant, anti-foam agent, thickener, polymeric binder, and/or solvent, and the composition may then be used for screen printing a pigment-layered substrate or a colored substrate.

Block 18 describes creation of a reservoir. The reservoir may be created on or in the substrate, e.g., a reservoir 130 shown in FIG. 1A. It will be appreciated that the reservoir may be provided during customization in order to permit the selection of an indicator material with a particular response, such as a particular melting point, and/or a particular viscosity. A gap in the light scattering layer may serve as a reservoir, or a reservoir may be created adjacent to the light scattering material on the substrate. In a different instance, one or more additional holders or containers may be provided. In some instances, creating a reservoir may involve overlaying the reservoir on the light scattering material (e.g., reservoir 330 shown in FIG. 4, reservoir 430 shown in FIG. 5, reservoir 530 shown in FIG. 6). After block 18, the first method 10 may be concluded.

The flowchart 20 illustrates a second method, for customizing an indicator, such as an indicator formed by performing the method 10.

Block 21 of the second method 20 describes creation of a boundary of a suitable shape and/or length. For example, a boundary 120 as shown in FIG. 1A may be created. The shape and length of the boundary may be created to accommodate a timer feature, for example, a boundary 220 as shown in FIG. 3.

Block 23 describes adding a selected indicator material to the reservoir. For example, a liquifiable material may be selected as described above for FIG. 2. A user may also select whether the indicator should be activatable at a time of a user′s choosing by selecting an appropriate reservoir material which only releases liquifiable material after an activation action, for example, as described above for FIG. 5 and FIG. 6.

Block 26 describes applying a sealing layer. This step is optional and may be omitted. A sealing layer may include a variety of materials that protect the covered layers and prevent penetration of humidity or gas, e.g., polymeric films made of, for example, polyethylene terephthalate (PET), or biaxially oriented polypropylene (BOPP).

Block 27 describes placing a cover on the indicator. This step is optional and may be omitted. If a cover is placed on the indicator, the cover may have one or more windows (e.g., as shown in FIG. 7). Multiple windows may be used where a timer feature is selected by a user to indicate progress of ingressed fluid through the light scattering material and thus, duration of exposure.

Block 28 describes applying print labeling to the indicator (e.g., “press here to activate”) as shown in FIG. 7. The print labeling may be applied through the cover layer if a cover layer was placed on the indicator. After block 28, the second method 20 may be concluded.

The flowchart 30 illustrates a third method, for deploying an indicator, such as an indicator such as an indicator formed by performing the first method, and/or an indicator customized by performing the second method.

Block 32 describes a user receiving a finished indicator, e.g., an indicator of FIG. 1A, FIG. 3, FIG. 4, FIG. 5 or FIG. 6 as described above.

Block 33 describes applying an optional activation action to the indicator. For example, a user may apply heat to the reservoir area before placing the indicator in the environment subject to testing. The heat may liquify the material in the reservoir, or may melt the shell of microcapsules releasing a liquid encapsulated in the microcapsules. In another example, an activation action may involve a user applying pressure to the reservoir area which may rupture a seal and expose material in the reservoir to humidity which will liquify the material in the reservoir. In a further example, an activation action may involve a user applying pressure to the reservoir area which causes microcapsules to burst and release liquid enclosed in the microcapsules. Applying an activation action is optional and depends on the reservoir and reservoir material included in the indicator, as customized in method 20.

Between block 33 and 35, an indefinite amount of time may pass. For instance, the liquified material from the reservoir may migrate through the channel. If a user selected an indicator that is activatable at a time of the user′s choosing, an activation action is applied to the reservoir and an indefinite amount of time may pass for the material in the reservoir to liquify and for the liquified material to flow through the channel.

Between block 33 and 35, an indefinite amount of time may also pass where the product is in use and vulnerable to exposure to environmental condition. In this period of time, the indicator may be associated with the host product which is being monitored.

Block 35 describes placing an indicator in a test environment so that the indicator is subjected to an environmental condition such as temperature, humidity or a gas.

Block 37 describes waiting for an indicator that has been placed in a test environment (with or without an optional activation action) to sense the environmental condition. In block 37, the amount of time that may pass may be pre-determined (i.e., the amount of time above a threshold environmental exposure which may be required to create an indication may be pre-determined).

Block 39 describes reading an indicum. An indicium may be read either by a human or by a machine. It will be appreciated that there may be a single indicium (e.g., “spoiled”), or there may be multiple indicia (e.g., a series of windows in the cover revealing indicia sequentially as the liquified material traverses the length of the indicator region, and the series of indicia revealed in the series of windows act as a timer).

In various examples the first method, the second method and the third method may be performed in sequence to manufacture, customize and deploy an activatable environmental exposure indicator, e.g., indicator 100, 200, 300, 400, or 500 of FIG. 1A, FIG. 3, FIG. 4, FIG. 5, and FIG. 6 respectively.

### Timer Feature

For the indicators 100, 200, 300, 400 and 500, the thickness of the light scattering layer which is overlayed on a colored substrate, or on a pigment layer overlayed on a substrate, is thick enough to mask the color of the pigment or colored substrate so that the light scattering material appears opaque or nearly opaque when the indicator has not been activated. The thickness of the light scattering layer may be increased as needed to accommodate a timer feature (e.g., in embodiments of FIG. 4, FIG. 5, or FIG. 6, where the reservoir is overlayed on the light scattering material). The length of the indicator region may be increased as needed to accommodate a timer feature (e.g., where the reservoir is in or on the substrate, by changing the shape of the indicator region by creating a suitable boundary as shown in FIG. 3).

For a timer feature, the viscosity of the liquid released from the reservoir and moving through the indicator region can be chosen so that the distance traveled by the fluid through the indicator region can be calibrated to duration of time that the indicator has exceeded a threshold temperature or humidity or gas.

### Activatable Indicators

For the indicators 100, 200, 300, 400, 500, and 600, in some embodiments, the material in the reservoir may liquify after an activation action. In some embodiments, it may be desirable that an indicator does not indicate until it is placed in a test location. In some embodiments, the reservoir material is covered with a seal. For example, to avoid accidental exposure to humidity, a user may break the seal on the reservoir after placing the indicator in a desired location. In some embodiments, the reservoir material has activatable microcapsules. For example, to avoid accidental exposure to temperatures in the indicating range, a user may rupture the activatable microcapsules by applying a compression force or by applying heat to the reservoir area after placing the indicator in a desired location.

In some embodiments, a user may needle the substrate after placing it in a desired location. In some embodiments, the microneedles are microneedles filled with microcapsules as shown in FIG. 6. Application of an activation action, e.g., a compression force, pushes the microcapsules into the indicator region. Also contemplated are embodiments where the microneedles are coated microneedles or dissolving microneedles which can release microcapsules into the indicator region. In some embodiments, the microneedles are hollow microneedles filled with microcapsules and the microcapsules can be pushed into the indicator region (e.g., by application of a compression force). The action of pushing the microcapsules out of the hollow microneedles may also cause the microcapsules to burst. Thus, needling delivers activatable microcapsules to the indicator region. The microcapsules are filled with liquid or a liquifiable material and may burst or melt in response to a compression force, or in response to a threshold temperature, humidity, or gas, thus delivering fluid to the indicator region, thereby exposing the indicium. In this manner, the transition of the light scattering layer from the obscuring form to the non-obscuring form may be delayed relative to the microcapsules rupturing and releasing liquifiable material.

### Material in Reservoir

In embodiments of indicators 100, 200, 300, 400, 500, and 600, the material in the reservoir is a liquifiable material. In some embodiments, the material in the reservoir may transition from a solid to a liquid (melt), or it may be gel-like and undergo a change in viscosity when exposed to a threshold temperature or humidity or gas, thus allowing the material to flow.

In some embodiments, the material in the reservoir is a liquifiable polymer or a wax, or a combination thereof, which can liquify and flow into the indicator region, thereby unmasking the indicium. The liquifiable polymer or wax, or combination thereof, in the reservoir may liquify when the temperature exceeds a threshold temperature. In some embodiments, the liquifiable polymer is a polyalkyl acrylate such as an SCC polymer described herein. SCC polymers have a conventional polymer backbone and side chains that can co-crystallize. Typically, they are chains that have six or more carbons with a crystallization temperature that is, therefore, adjustable. An advantage of SCC polymers is that their molecular weight and degree of crosslinking can be adjusted to control their physical properties including their melting temperatures and/or viscosities, providing an approach to tailor the timer function of the indicators described herein. In this manner, the SCC material is variably configurable. In some embodiments, an SCC has C₁₀-C₃₀ sidechains, or C₁₄-C₂₂ side chains. In some embodiments, the molecular weight of an SCC used as reservoir material ranges from about 1,000 to about 400,000 Da. With regard to SCC materials in particular, the melting point of SCC materials may be tuned by increasing the side-chain length of the molecules of the SCC material, and the viscosity (when liquefied) may be tuned by increasing the quantity of side chains of the molecules of the SCC material, thus increasing the molecular weight.

The SCC polymer may be a homopolymer or a copolymer. The SCC polymer′s melt temperature is variably configurable by selecting a suitable side chain length. The side chains in the SSC polymer are selected to form crystalline regions and may include, for example, —(CH₂)n— and/or —((CH₂)ₘm—O—)ₙ groups. The side chains may be linear to facilitate crystallization. For SCC polymers that contain —(CH₂)ₙ— groups in the crystallizable side chain, n may be in the range of about 4 to about 22, or from about 4 to about 16. For SCC polymers that contain —((CH₂)ₘ—O—)n groups in the crystallizable side chain, m may be in the range of about 1 to about 20, or about 1 to about 10. In some embodiments, m and n are selected and the spacing between side chains and the length and type of side chain are selected to provide a SCC polymer with a desired melting point. In some embodiments, the length of the crystallizable side chain may be in the range of about two times to about ten times the average distance between crystallizable side chains in the heat-sensitive polymer. Examples of SCC polymers include and are not limited to poly(1-alkene)s, poly(alkyl acrylate)s, poly(alkyl methacrylate)s, poly(alkyl vinyl ether)s, and/or poly(alkyl styrene)s, or any combination thereof. In some embodiments the side chains in SCC polymers have from 10 to 30, or 14 to 22 carbon atoms. Non-limiting examples of SCC polymers include poly(dodecylacrylate), poly(tetradecylacrylate), poly(hexadecylacrylate), poly(octadecylacrylate), copolymer of hexylacrylate and dodecylacrylate, copolymer of hexylacrylate and docosylacrylate, copolymer of decylacrylate and tetradecylacrylate, copolymer of decylacrylate and octadecylacrylate, copolymer of decylacrylate and octadecylacrylate, copolymer of decylacrylate and octadecylacrylate, copolymer of dodecylacrylate and docosylacrylate, copolymer of dodecylacrylate and docosylacrylate, copolymer of dodecylacrylate and docosylacrylate, copolymer oftetradecylacrylate and octadecylacrylate, copolymer oftetradecylacrylate and octadecylacrylate, copolymer oftetradecylacrylate and octadecylacrylate, poly(dodecylmethacrylate), poly(tetradecylmethacrylate), poly(hexadecylmethacrylate), poly(octadecylmethacrylate), copolymer of tetradecylmethacrylate and methyl methacrylate, copolymer of octadecylmethacrylate and methyl methacrylate.

In addition to SCC polymers, suitable liquefiable materials for the disclosed applications may include non-SCC polymers, polymeric waxes, synthetic waxes, natural waxes and combinations thereof.

In some embodiments, alkane waxes include paraffin waxes which melt above a temperature threshold (e.g., about 40° C.). In some embodiments, alkanes having more than 18 carbon atoms are typically solids which can liquify above a temperature threshold. For example, tetracontane (C₄₀H₈₂) available from ThermoFisher has a reported melting point of about 80° C. to 83° C. In this manner, the threshold temperature for the material in the reservoir can be variably configured.

In some embodiments, a first liquefiable material may be combined with a second liquefiable material to form a combined liquefiable material which has a liquefication point distinct from the liquefication points of either the first or second liquefiable materials.

Various liquefiable materials suitable for use in the disclosed applications may have melting points or liquefaction points across a broad range of temperatures. Useful liquefiable materials may have a melting point or liquefaction point between 0 degrees C. and 100 degrees C., although temperatures above 100 degrees C. and below 0 degrees C. are also contemplated.

In some embodiments, the material in the reservoir may be activatable microcapsules that release liquid or liquifiable material in response to an activation action (e.g., application of compressive force, heat exceeding a temperature threshold).

The indicators described herein may utilize microcapsules having frangible shells encapsulating liquids or liquifiable material (e.g., hygroscopic material, polymers (e.g., SCC polymers). alkane wax). The frangible shells are rupturable, e.g., the frangible shells rupture and release liquid when subjected to an activation action, rendering the indicator responsive to an environmental stimulus.

The microcapsules may be any size, but in one embodiment, may have an outer diameter length between 20-1000 μm. A variety of microcapsule shell materials may be chosen, depending on the application, the mode of rupture, and the nature of the contents of the microcapsule. In general, the microcapsules should resist the passage, whether by flow, diffusion, or migration, of the contents of the microcapsule prior to rupturing.

The microcapsule is initially in an unruptured form, capable of being configured to transition to a ruptured form through exposure to an activation action, e.g., the application of heat, pressure, and/or a combination of heat and pressure exceeding a predetermined threshold. In the unruptured form, the frangible shell of the microcapsule maintains separation between the contents of the microcapsule and any external environmental stimuli such as humidity or gas. The microcapsule may rupture in various modes. A first rupture mode is rupture or weakening by externally applied pressure, i.e., where the activation action is an exposure to a compressive or shearing force. A second rupture mode is rupture or weakening by heat exposure, i.e., where the activation action is an exposure to a temperature configured to melt, degrade, decrease the structural integrity of, or otherwise disengage the frangible shell. A third rupture mode is rupture or weakening via an internally applied pressure. In some such examples, the microcapsules may be ruptured or weakened by a source of internal pressure, where the activation action is configured to trigger expansion of a material within the frangible shell (e.g., a volatile material, thermally expandable microsphere) which increases the internal pressure of the microcapsule, which ruptures or weakens the frangible shell. Generally, materials suitable for forming frangible shells which are configured to rupture may include polymers, elastomers, inorganics, or combinations thereof.

In some embodiments, the shell of the microcapsule may be a single shell made of a porous polymer and a waxy material or a clay that seals the pores in the polymer making the microcapsule impermeable to humidity or gas till it ruptures (e.g., by application of a compressive force or by melting after a threshold temperature is reached). The shell of the microcapsule may, in an alternate embodiment, be a dual shell. An inner shell made of a porous polymer is then coated with a waxy material or a clay that seals the pores in the polymer making the microcapsule impermeable to humidity or gas till it ruptures (e.g., by application of a compressive force or by melting after a threshold temperature is reached). In some embodiments, the shell of a microcapsule may be Expancel®, an SCC polymer, an alkane wax, a kaolin clay, or combinations thereof.

### Light Scattering Material

The light scattering material which is overlayed on a colored substrate, or on a pigment layer which is overlayed on the substrate, may be crystalline or semicrystalline or may be a flocculate. In some embodiments, the light scattering material has particles of a suitable polymer where there are spaces or voids between the particles and the particles are porous.

As used herein, “scattering” may describe a wide range of physical processes where moving particles (e.g., photons) or radiation of some form (e.g., light), is forced to deviate from a straight trajectory by localized non-uniformities (including particles or voids) in the medium through which they pass. Deviations in the trajectory may also be caused by reflections of radiation (e.g., light). Reflections of radiation that undergo scattering are often does called diffuse reflections and unscattered reflections are called specular (mirror-like) reflections. Without being bound to a particular theory, at an atomic level, “scattering” refers to particle-particle collisions between molecules, atoms, electrons, photons and other particles. In the illustrated example, “scattering” may be caused by non-uniformities, such as voids in the light scattering material. The non-uniformities may exist on the light scattering material's surface, within the light scattering material, or a combination thereof. However, the types of non-uniformities which can cause scattering, sometimes known as “scatterers” or “scattering centers”, may include particles, bubbles, droplets, density fluctuations in fluids, crystallites in polycrystalline solids, defects in monocrystalline solids, surface roughness, cells in organisms, textile fibers in clothing among others. The effects of such features on the path of almost any type of propagating wave or moving particle can be described in the framework of scattering theory. In several of the examples described herein, the “scatterers” are voids, which may be voids within particles of material, voids between particles of material, voids in the surfaces of particles of material, voids within a solid mass of material, or spaces formed between particles of material.

Generally, when light is passing through the light-scattering material, the voids (along with other non-uniformities) may cause the light to deviate from a straight trajectory and the aggregate effect of each instance of scattering causes the light-scattering material to be non-transparent or opaque. When the light scattering material appears opaque, the light scattering material is in the obscuring form, and may be used to obscure object or indicia from view.

When in bulk form, (e.g., homogenized so as not to include voids) the base materials used to form light scattering materials may be generally transparent (e.g., non-light scattering), and are subsequently processed into a form which is light scattering. The light scattering layers of the present disclosure are generally formed of a transparent material which has been particlized and compounded into a layer where voids exist between the particles of transparent material. The spaces between the particles of transparent material provide surfaces within the layer for incident light to scatter off of, which at scale, renders the light scattering material to appear opaque.

For use in the disclosed applications, light scattering layers must also be convertible or otherwise transformable to a non-obscuring, or non-light scattering state. In some examples, the light scattering layer may be acted upon by a compressive force, a thermal input, or some other external action which causes, by compression, melting, or other action, the voids in the light scattering material to collapse. In other examples, the voids of the light scattering material may be filled with a liquid, or liquefiable material, which is transparent, the filling of the voids causing the light scattering effect to abate, such that the light scattering layer transitions to the non-obscuring form.

In the above-described manner, light scattering materials may be used to initially obscure objects or indicia from a viewer, and later reveal the initially obscured object or indicia when the voids of the light scattering material are collapsed or filled.

The light scattering material may include a uniform arrangement of voids that are evenly distributed throughout the material, or allocated towards certain features, such as a surface, of a light scattering layer.

An example of a light scattering layer may include a polymer which is particlized and physically compounded to form a light scattering material. Some such suitable polymers include Polytetrafluoroethylene (PTFE), Ultra High Molecular Weight Polyethylene (UHMW PE), Ultra High Molecular Weight Polypropylene (UHMW PP), High Density Polyethylene (HDPE) Polyvinvylidene Fluoride (PVDF), Perfluoroalkoxy alkane (PFA), Liquid Crystalline Polymer (LPC). In some examples, the polymer which forms the light scattering layer is a side-chain crystalline polymer. In some embodiments, the light scattering layer may have particles of SCC or LCP polymers.

In some embodiments, Sidechain Crystalline Polymers (SCCs) in light scattering layers provide enhanced refractive index and structural properties that improve light scattering capabilities. In some embodiments, light scattering layers may include titanium dioxide (TiO₂), zinc oxide (ZnO), calcium carbonate (CaCO3), polymeric microspheres made from polystyrene or PMMA, hollow sphere pigments, silica, alumina (aluminum oxide), nanosilver, nanogold, quantum dots, cholesteric liquid crystals, cellulose nanocrystals, starch derivatives, blends of organic and inorganic materials, barium sulfate (BaSO₄), magnesium carbonate (MgCO₃), talc, kaolin clay, mica, titanium oxynitride (TiON), yttrium oxide (Y₂O₃), cerium oxide (CeO₂), antimony-doped tin oxide (ATO), indium tin oxide (ITO), zirconia (ZrO₂), boron nitride (BN), hexagonal boron nitride (h-BN), lanthanum oxide (La₂O₃), and/or gadolinium oxide (Gd₂O₃).

Without being bound by theory, light scattering may happen when there is a variation in the refractive index caused by particles or voids. In some embodiments, a light scattering layer includes emulsion-based SCC polymer particles ranging from about 400 to about 700 nm in size, along with air voids between these particles. The polymer particles can melt and coalesce into a clear, transparent film.

In some embodiments the micro-voids or micro-cavities in a light scattering layer may be created through processes using calcium carbonate, and then stretching the film to create a film of low density.

An example of a light scattering layer may include a micropillar array film, a nanopillar array film, or the like. The films may be prepared by laser etching the film to create an appropriate surface profile including the voids. In another example, the light scattering material may be prepared by chemical deposition.

### Customization

The light scattering material and the material in the reservoir may be selected as described above. Depending on the user′s requirements, the boundary region is created so as to accommodate a desired length or thickness or shape of the indicator region. Depending on the flow rate/viscosity of the liquid flowing from the reservoir to the indicator region, the length or thickness of the light scattering material in the indicator region can be varied and calibrated to provide a timer function. In this manner, a fully customizable indicator label can be provided to a user.

### EXAMPLES

### Example 1

A first portion of a substrate is coated with a dark pigment, overlayed with particles of a commercially available C₁₄-C₂₂ SCC polymer by screen printing, and then thermally printed to melt portions of the SCC polymer layer and provide a boundary region as shown in FIG. 8. The SCC polymer is light scattering and hides the dark color of the underlying pigmented substrate. A second portion on the substrate is covered with paraffin wax and a sealing film to provide a reservoir.
FIG. 8 shows four panels. Panel A is a substrate layered with light scattering material and having a boundary created thereon. The region within the boundary is in fluid contact with a reservoir of solid paraffin wax. Panel B is a substrate layered with light scattering material, but no boundary created thereon, and in fluid contact with a reservoir of solid paraffin wax within a pencil-drawn circle as shown.

When the temperature exceeds a threshold temperature, the paraffin wax melts, the liquid flows into the light scattering material and the light scattering material becomes transparent, revealing the pigment underneath (as shown by a color change from white to black).

In panel A′ the boundary region confines the fluid flow to within the boundary. In panel B′, which lacks a boundary, the liquid flow is outside of the pencil-drawn circles.

This experiment illustrates the working of the indicators described herein. The experiments shows that a light scattering material can be first subjected to melting in selected areas to provide a boundary, that the boundary defines an impermeable barrier, and that fluid flow to the indicator region as defined by the boundary would be limited to the area defined by the boundary.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. Additionally, the described embodiments/examples/implementations should not be interpreted as mutually exclusive, and should instead be understood as potentially combinable if such combinations are permissive in any manner. In other words, any feature disclosed in any of the aforementioned embodiments/examples/implementations may be included in any of the other aforementioned embodiments/examples/implementations.

The benefits, advantages, solutions to problems, and any element(s) that may cause any benefit, advantage, or solution to occur or become more pronounced are not to be construed as a critical, required, or essential features or elements of any or all the claims. The claimed invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The term “coupled” as used herein is defined as connected, although not necessarily directly and not necessarily mechanically. A device or structure that is “configured” in a certain manner is configured in at least that manner, but may also be configured in manners that are not listed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separately claimed subject matter.

The invention is claimed as follows:

1. A method of making an environmental indicator, the method comprising:
providing a substrate;
providing an indicia in an indicator region on the substrate;
providing a reservoir on or in the substrate;
overlaying cavitated porous light scattering material on the substrate, the light scattering material masking the indica;
closing the pores of the cavitated porous light scattering material in a boundary region by heating portions of the cavitated porous light scattering material,
the boundary region defining a channel through the porous light scattering material between the reservoir and the indicator region;
and
adding indicator material to the reservoir,
the indicator material configured to liquify responsive to a predetermined environmental stimulus and flow through the channel to the indicator region filling the cavities in the cavitated porous light scattering material in the indicator region and thereby unmasking the indicia.

2. The method of claim 1, wherein the material in the reservoir includes a component selected from the group consisting of a polyalkyl acrylate, an alkane wax, and combinations thereof.

3. The method of claim 2, wherein the polyalkyl acrylate is a side chain crystalline (SCC) polymer having C₁₀-C₃₀ alkyl side chains.

4. The method of claim 2, wherein the alkane wax comprises a C₁₈-C₄₀ hydrocarbon, or a mixture of C₁₈-C₄₀ hydrocarbons.

5. The method of claim 1, wherein the material in the reservoir comprises a plurality of microcapsules which include a liquid or a liquefiable material microencapsulated in a frangible shell, wherein the frangible shells are configured to release the liquid or the liquefiable material responsive to an application of an activation action.

6. The method of claim 5, wherein an activating action is application of a compressive force or application of heat to the reservoir.

7. The method of claim 1, further comprising:
a plurality of needles configured to penetrate the channel, placing the reservoir into fluid communication with the channel.

8. The method of claim 7, wherein the needles are hollow and the material in the reservoir flows through the needles.

9. The method of claim 1, wherein an indicium is a machine-readable indicium, which is readable through the cavitated porous light scattering material after the liquefiable material fills the pores in the channel and is unreadable through the cavitated porous light scattering material before the liquefiable material fills the pores in the channel.

10. The method of claim 1, wherein the environmental stimulus is selected from the group consisting of an exposure to an ambient temperature above a predetermined high temperature threshold, an exposure to an ambient temperature above a predetermined high temperature threshold for at least a predetermined amount of time, a cumulative exposure to ambient heat exceeding a predetermined cumulative heat threshold, a cumulative exposure to humidity exceeding a predetermined cumulative humidity exposure threshold, and a cumulative exposure to a gas exceeding a predetermined cumulative gas exposure threshold.

11. The method of claim 1, wherein the cavitated porous light scattering material comprises a material selected from a side chain (SCC) crystalline polymer, Polytetrafluoroethylene (PTFE), Ultra High Molecular Weight Polyethylene (UHMW PE), Ultra High Molecular Weight Polypropylene (UHMW PP), High Density Polyethylene (HDPE), Polyvinylidene Fluoride (PVDF), Perfluoroalkoxy alkane (PFA), Liquid Crystalline Polymer (LCP), or combinations thereof.

12. The method of claim 1, wherein the cavitated porous light scattering material comprises a side chain crystalline polymer.

13. The method of claim 1, wherein the cavitated porous light scattering material comprises particles having an average diameter ranging from about 200 nm to about 800 nm.

14. The method of claim 1, wherein the cavitated porous light scattering material comprises particles having an average diameter ranging from about 400 nm to about 600 nm.

15. The method of claim 1, wherein portions of the cavitated porous light scattering material are heated, thereby closing the pores of the cavitated porous light scattering material in a boundary region.

16. The method of claim 15, wherein portions of the cavitated porous light scattering material are heated to temperatures ranging from about 40° C. to about 300° C.

17. An environmental indicator comprising:
a substrate;
an indicia in an indicator region on the substrate;
a layer of overlay material overlaying the substrate,
a first portion of the layer of overlay material over the indicator region being cavitated, porous and light scattering, and masking the indicia;
a second portion of the layer of overlay material being cavitated, porous and light scattering, and forming a channel connecting the indicator region with the reservoir;
a third portion of the layer of overlay material having closed pores to define the boundaries of the channel;
and
a reservoir of indicator material;
the indicator material configured to liquify responsive to a predetermined environmental stimulus and flow through the channel to the indicator region filling the cavities in the cavitated porous light scattering material in the indicator region and thereby unmasking the indicia.

18. The environmental indicator of claim 17, wherein the material in the reservoir includes a component selected from the group consisting of a polyalkyl acrylate, an alkane wax, and combinations thereof.

19. The environmental indicator of claim 18, wherein the polyalkyl acrylate is a side chain crystalline (SCC) polymer having C₁₀-C₃₀ alkyl side chains.

20. The environmental indicator of claim 18, wherein the alkane wax comprises a C₁₈-C₄₀ hydrocarbon, or a mixture of C₁₈-C₄₀ hydrocarbons.

21. The environmental indicator of claim 17, wherein the material in the reservoir comprises a plurality of microcapsules which include a liquid or a liquefiable material microencapsulated in a frangible shell, wherein the frangible shells are configured to release the liquid or the liquefiable material responsive to an application of an activation action.

22. The environmental indicator of claim 21, wherein an activating action is application of a compressive force or application of heat to the reservoir.

23. The environmental indicator of claim 17 further comprising:
a plurality of microneedles configured to penetrate the channel, placing the reservoir into fluid communication with the channel.

24. The environmental indicator of claim 23, wherein the needles are hollow and the material in the reservoir flows through the needles.

25. The environmental indicator of claim 17, wherein an indicium is a machine-readable indicium, which is readable through the cavitated porous light scattering material after the liquefiable material fills the pores in the channel and is unreadable through the cavitated porous light scattering material before the liquefiable material fills the pores in the channel.

26. The environmental indicator of claim 17, wherein the environmental stimulus is selected from the group consisting of an ambient temperature above a predetermined high temperature threshold, an exposure to an ambient temperature above a predetermined high temperature threshold for at least a predetermined amount of time, a cumulative exposure to ambient heat exceeding a predetermined cumulative heat threshold, a cumulative exposure to humidity exceeding a predetermined cumulative humidity exposure threshold, and a cumulative exposure to a gas exceeding a predetermined cumulative gas exposure threshold.

27. The environmental indicator of claim 17, wherein the cavitated porous light scattering material comprises a material selected from a side chain crystalline polymer, Polytetrafluoroethylene (PTFE), Ultra High Molecular Weight Polyethylene (UHMW PE), Ultra High Molecular Weight Polypropylene (UHMW PP), High Density Polyethylene (HDPE) Polyvinylidene Fluoride (PVDF), Perfluoroalkoxy alkane (PFA), Liquid Crystalline Polymer (LCP), or combinations thereof.

28. The environmental indicator of claim 17, wherein the cavitated porous light scattering material comprises a side chain crystalline polymer.

29. The environmental indicator of claim 17, wherein the cavitated porous light scattering material comprises particles having an average diameter ranging from about 200 nm to about 800 nm.

30. The environmental indicator of claim 17, wherein the cavitated porous light scattering material comprises particles having an average diameter ranging from about 400 nm to about 600 nm.

31. The environmental indicator of claim 17, wherein portions of the cavitated porous light scattering material are heated, thereby closing the pores of the cavitated porous light scattering material to define the boundaries of the channel.

32. The environmental indicator of claim 31, wherein portions of the cavitated porous light scattering material are heated to temperatures ranging from about 40° C. to about 300° C.

33. An environmental indicator produced using the method of claim 1.
